(19) United States
(12) Patent Application Publication
(10) Pub. No.: US 2005/0159754 A1
(43) Pub. Date: Jul. 21, 2005

(54) Periosteal distraction bone growth

(76) Inventor: Ronald B. Odrich, New York, NY (US)

Correspondence Address: NOTARO AND MICHALOS, 100 DUTCH HILL ROAD, SUITE 110, ORANGEBURG, NY, 10962-2100, US

(21) Appl. No.: 10/761,840

(22) Filed: Jan. 21, 2004

(51) Int. Cl. A61F 5/04; A61F 5/00; A61B 17/56

(52) U.S. Cl. 606/86; 606/53

(57) ABSTRACT

A periosteal distraction osteogenesis method and apparatus uses a sheet member for covering a surface of living bone that is, in turn, covered by soft tissue. The sheet member is under the soft tissue and over an area where bone growth outwardly and normally to the bone surface is desired. An attractor member adapted to magnetically attract the sheet member for exerting a pulling force on the sheet member in a direction outwardly and normally of the bone surface, is secured at an outwardly spaced location from the sheet member for causing growth of bone outwardly and normally to the bone surface.

### FIELD AND BACKGROUND OF THE INVENTION

The present invention relates generally to the field of osteogenesis (bone growth), and in particular to a new and useful method and apparatus for growing bone in selected areas of a human or animal subject, particularly, but not exclusively, for growing bone at the crest of the jawbone.

It is often desirable to encourage bone to grow. Any bone fracture to be treated, for example, relies on bone grow to knit the bone back together in the fractured area.

In the mouth, when a tooth or teeth are lost, the jawbone in the edentulous area tends to shrink. This poses a particular problem in cases where implants are being considered for replacing the missing tooth or teeth, since a certain minimum amount of bone mass and depth is needed. It has not been possible until now to simply induce a jawbone to grow occlusally (outwardly) from the crest of the bone, that is, in a direction normal to the outer surface of the bone, without involving complex procedures using grafting materials, membranes, etc.

U.S. Pat. No. 4,871,310 to Vardimon teaches a magnetic appliance that corrects skeletal malocclusions. The appliance includes an upper magnetic unit secured to the upper jaw and a lower magnetic unit secured to the lower jaw. The units can be placed labial or lingual to the dental arch. When the mouth is closed, the magnetic units come into contact with each other, thereby pulling the mandibular jaw toward the maxillary jaw and reducing the skeletal imbalance via the guidance of jaw growth. Although this patent teaches the pulling of jawbone for guiding jawbone growth, it fails to teach a magnetically reactive material embedded subperiostally and does not induce occlusal bone growth normally, that is perpendicularly, to the bone surface. The patent rather teaches guidance of the ususal type of bone growth during a ususal period of accelerated growth.

U.S. Pat. No. 4,484,895 to Smiley et al. discloses an intra-oral positioning fixture for orthodontic and periodontal therapy and bone augmentation. The fixture includes a plastic or elastomeric material with flanges that define a channel and conform to the teeth and periodontium. Sources of magnetic flex, such as permanent magnets, are embedded in both the buccal and lingual flanges and are magnetically coacting via either attraction or repulsion forces. This patent does not teach a magnetically reactive material embedded subperiostally for inducing outward jawbone growth.

U.S. Pat. No. 6,398,713 to Ewing et al. discloses a mouthpiece comprising a bite plate and at least one embedded magnetic device. The magnetic device, such as a magnet, is oriented within the mouthpiece to optimally expose joints, bones and surrounding tissue to a magnetic field. This patent also discloses a magnetic dental cement which exposes the tooth and surrounding soft tissue to the effects of a magnetic field, positively impacting blood circulation and cell polarity to reduce pain and promote healing according to the reference. It fails to teach a magnetically reactive material embedded subperiostally for inducing growth of the jawbone.

U.S. Pat. No. 6,032,677 to Blechman discloses a dental implant that can produce bone growth toward the implant from the implant-bone interface. The implant is connected to a magnet that is oscillated by an externally applied, time varying moving magnetic field. Movement of the magnet connected to the dental implant translates to micromotion of the implant, which stimulates bone growth at the implant bone interface according to Blechman, but no occlusal growth is shown or contemplated.

A variety of patents teach magnetic appliances for corrective movement of teeth. For example, U.S. Pat. No. 4,424,030 to Smiley et al. discloses a magnetic appliance having one magnetic unit attached to an upper arch wire secured to the upper jaw and another magnetic unit attached to a lower arch wire secured to the lower jaw. The magnetic units create a magnetic force which causes tooth movement. Movement of the magnetic units during mandibular movement produces bone formation in void areas caused by tooth movement. U.S. Pat. Nos. 4,595,361 and 4,671,767 to Blechman et al. disclose a magnetic force orthodontic appliance which contains permanent magnet modules supported on attachment wires coupled to teeth of the maxillary and mandibular arches. The magnetic modules can be combined in various combinations relying on the attraction between unlike poles or repulsion between like poles. The modules can be oriented with either pole face positioned to cooperate with an adjacent magnet. The confrontation of the magnet modules exerts a force to induce malocclusion correction.

General tissue growth via magnetic application is also known. U.S. Pat. No. 5,458,558 to Liboff et al. teaches a method and apparatus for controlling tissue growth via the application of a controlled, fluctuating, and directionally oriented magnetic field. The method is applicable to bone growth among other types of tissue growth, but not for bone growth that is normal to, and outward of the bone surface.

U.S. Pat. No. 6,179,871 to Halpern teaches a method for cartilage repair in which holes are drilled at the base of a cartilage defect, a porous scaffold material containing magnetic particles is deposited in the holes, and a magnetically tagged growth promoting material is injected into the area of the defect. The magnetically tagged growth promoting material is then drawn into the holes by the magnetic particles contained within the holes. The migrating growth factors promote chondrogenesis within the defect.

U.S. Pat. No. 4,175,565 to Chiarenza, et al. discloses a method and apparatus for stimulating osteogenic activity in a jawbone to anchor a dental implant. A direct current is applied to the implant acting as a cathode and a second electrode acts as an anode.

There is no teaching in the prior art of any method or apparatus for inducing outward bone growth that is normal, that is, perpendicular, to the outer bone surface using magnetic attraction.

### SUMMARY OF THE INVENTION

The periosteal distraction osteogenesis (bone growth) method and apparatus of the present invention comprise the application of an external magnet or attractor member to a magnetically reactive material that is embedded underneath soft tissue (e.g. skin, gingival or gum tissue, etc.) but placed over the bone surface (e.g. jawbone, femur, cranial bones, ulna, etc.). The application of the magnet results in a pulling force on the magnetically reactive material in an outward direction from the external surface of the bone (e.g. at the crest of the jawbone or the cortex of other bony structures of the body). The pulling force of the magnetically reactive material thereby stimulates the formation of bone at the periosteal or cortical surface of the bone.

In the case of oral application, the bone grows occlusally, that is in a perpendicular and laterally, normal or outward direction with respect to the crest or outer jawbone surface. The perforation of the cortical plate in the case of long bones or other bones such as cranio-facial bones or jawbones is also part of the invention in order to enhance the process by involving endosseous mesenchymal calls.

The same technique is applied to raise the floor of the maxillary sinus or to encourage the growth or lengthening of long bones or digits (toes or fingers).

The magnetically reactive material may be a wire mesh, a perforated or solid plate or any such structure which is made of, or includes, one of the magnetically attracted metals, iron, nickel and/or cobalt. The mesh or structure may have a variety of shapes and configurations to increase the effectiveness of the magnet on the mesh or structure. For example, the mesh/structure may have a thicker central portion to concentrate the pulling force of the magnet on a particular area of the bone, which can compensate for the shape of alveolar or other bony defect or deficiency.

In the case of a dental application, the magnet is either fixed between teeth or removable. The magnet may also be shaped differently to increase the effectiveness of the pulling force on the mesh/structure, especially if a specific form of bone growth is desired.

The mesh/structure may be made of a magnetically reactive material and comprise a loose link mesh capable of expanding in all directions or so structured as to expand in such a way as to create a desired form, e.g. a saddle shape. This would include vertical growth, horizontal growth and growth in any direction (i.e. on facial bones). This aides in producing a shape on the bone that achieves the desired result, namely growth outwardly and generally normal to the surface plane of the bone on which the mesh or structure lays.

The borders of the mesh may be tacked down with tacks around the perimeter or otherwise stabilized in order to anchor the edges of the mesh/structure while its central corpus is being pulled toward the magnet.

Alternatively, the mesh/structure, which will hereafter be called a sheet member to evoke its thin, area covering characteristic, may be made of magnetic material and the attractor member spaced outwardly of the sheet member may be magnetically attracted material (i.e. iron, nickel of cobalt). In a further alternative, both sheet member and attractor member may be magnetic material with opposite poles facing each other for maximum pulling force away from the surface of the bone.

The magnet or magnets (if a series of magnets is indicated) forming the attractor member, may be either fixed to adjacent structures or it/they may be removable. In the case of an oral application, fixation to the teeth may be possible or teeth may be used to support a removable magnet or an appliance containing a magnet or magnets.

The method and apparatus of the present invention may be applied to any instance where bone growth is indicated anywhere in the body. For example, it may apply in the field of Orthopedics where additional bone length or width of a bone or bones is desired, i.e. the hands, legs, fingers, feet, facial bones, etc. It may be applied to increase the height of an individual where arrested growth or other problem of development exists. It may do so, among other possibilities, by being applied to increase the length of long bones. The method may also be applied to cranio-facial deficiencies or deformities and any condition requiring the growth of bone.

Accordingly, an object of the present invention is to provide a periosteal distraction osteogenesis method and apparatus that uses a sheet member for covering a surface of living bone that is, in turn, covered by soft tissue. The sheet member is under the soft tissue and over an area where bone growth outwardly and normally to the bone surface is desired. An attractor member adapted to magnetically attract the sheet member for exerting a pulling force on the sheet member in a direction outwardly and normally of the bone surface, is secured at an outwardly spaced location from the bone surface and the sheet member, for causing the growth of bone outwardly and normally to the bone surface.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a side view of a human jawbone partly covered by soft tissue including gingival tissue (gum) and with an edentulous or tooth-less area between two teeth where the bone has receded as is common;

FIG. 2 is a view similar to FIG. 1 illustrating a first sequence of the method of the invention, using apparatus of the invention;

FIG. 3 is a view similar to FIG. 1 illustrating a further phase of the method of the invention, again using apparatus of the invention;

FIG. 4 is a view similar to FIG. 1 illustrating a still further phase of the method of the invention, using apparatus of the invention;

FIGS. 5, 6, 6A, 7 and 8 are respective top plan views of examples of magnetically attractable sheet members of the invention;

FIG. 9 is a partial top plan view of a jawbone illustrating a further embodiment of the apparatus of the invention;

FIG. 10 is a view similar to FIG. 1 illustrating the embodiment of FIG. 9;

FIG. 11 is an enlarged view of another embodiment of the apparatus of the invention;

FIG. 12 is a schematic side view of the head of a subject with a receding chin who can benefit from the present invention;

FIG. 13 is an enlarged side view of an apparatus according to the invention for improving the receding chin shown in FIG. 12;

FIG. 14 is a schematic view of the hand of a subject who has a finger stump;

FIG. 15 is an enlarged side view of an apparatus according to the invention for treating the finger stump shown in FIG. 14;

FIG. 16 is a schematic view of the leg of a subject with a serious fracture; and

FIG. 17 is an enlarged side view of an apparatus according to the invention for treating the fracture shown in FIG. 16.

### DESCRIPTION OF THE PREFERRED EMBODIMENTS

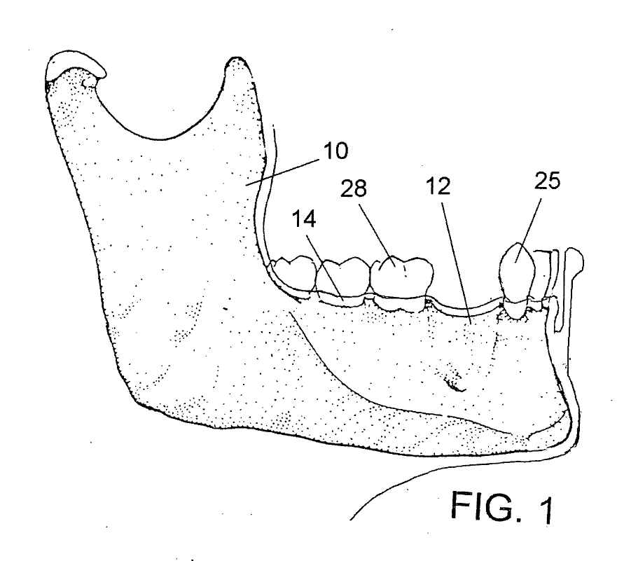

Referring now to the drawings, in which like reference numerals are used to refer to the same or similar elements, FIG. 1 illustrates a jawbone 10 with some teeth, in particular tooth number 25 and tooth number 28 having an edentulous or toothless area 12 therebetween. Gingival tissue or gum 14 covers the jawbone and edentulous area and surrounds the teeth in usual fashion. As is know, the jawbone in area 12 tends to shrink or recede after the teeth (numbers 26 and 27, not shown) have been lost. If a dental implant is desired in area 12 to replace the missing teeth, however, a certain minimum amount of bone must be present.

According to the present invention, bone can be induced to grown occlusally, that is outwardly and normally to the crest surface of the jawbone.

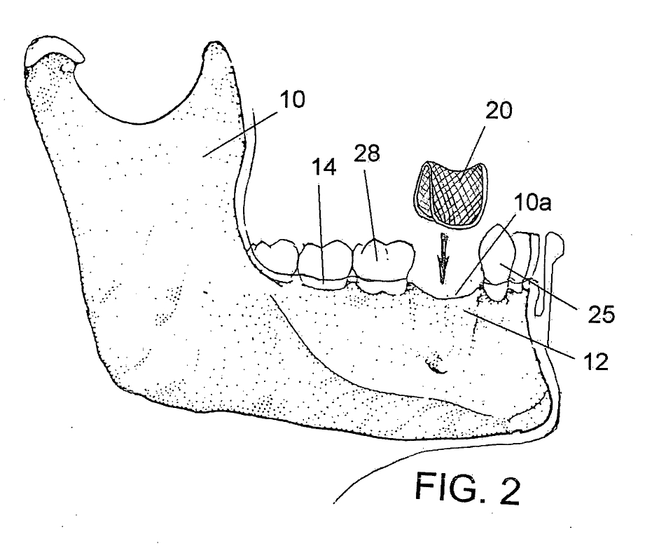

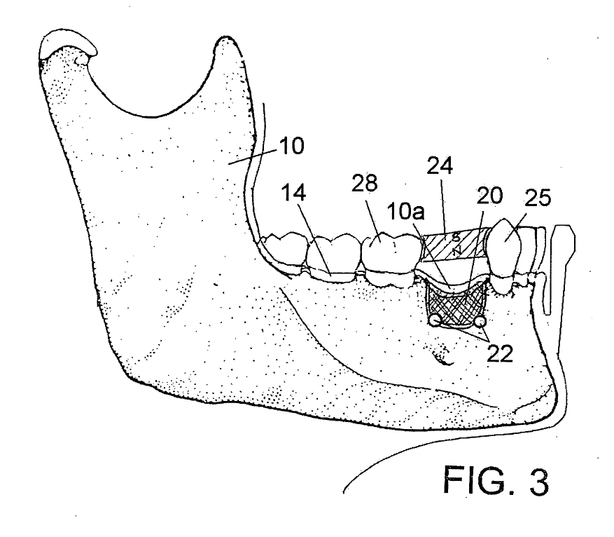

Referring to FIG. 2, the periosteal distraction osteogenesis method of the invention comprises cutting and temporally lifting a flap of gingiva from the edentulous area 12 to expose a portion 10a of the jawbone where occlusal bone growth is desired. This is a surface of living bone where the upper layers of cells are capable of growth if appropriately stimulated. According to the invention a sheet member 20 which is formed into an shape corresponding to the bone surface at 10a, e.g. a saddle shape, is placed onto the bone surface. As shown in FIG. 3, tacks 22 or other appropriate means are used to hold outer margins of the sheet member 20 to the jawbone 10, while leaving the portions of the sheet member that is over the bone surface portion at 10a where growth is needed, free.

The soft gum tissue is then folded back over the sheet member 20 on the bone surface at 10a and sutured in place. An attractor member 24, in the form of a permanent magnet, is then secured at an outwardly spaced location from the surface portion of bone at 10a, at which growth is desired. Attractor 24 magnetically attracts the sheet member which is made of magnetically attractable material such as sterilizable iron. The magnetic attractor member 24 applies a pulling force on the sheet member 20 that is away from the portion 10a of the bone surface at which bone growth is desired for causing growth of bone outwardly and normally to the bone surface. The pulling force need not, and in fact, should not physically lift the sheet member 20 and the gum tissue thereon, away from the bone surface at 10a, but rather the pulling action will stimulate the bone growing cells in the outer layer of bone tissue to grown to try and relieve the outward pressure caused by the magnetic attraction of the sheet 20 to the attractor member 24.

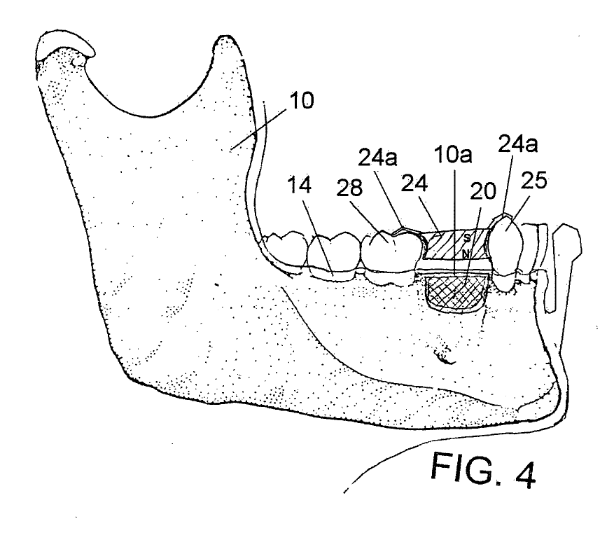

After about two to six weeks in place, the apparatus of the invention will cause bone to grow outwardly and normally toward the attractor member 24 as shown in FIG. 4, so that sufficient jawbone depth will be available for receiving a dental implant in tooth-less area 10a. Side magnets may also be provided to induce thickening of the jawbone in area 10a if desired, by pulling at the sides of the magnetically attractable mesh of sheet member 20.

FIG. 4 also illustrates means for securing the attractor member 24 to the adjacent teeth 25, 28, namely a pair of clamps or bands 24a for engaging over or around the adjacent teeth 25 and 28.

FIGS. 3 and 4 illustrate the desirability of orienting the poles of the magnet forming attractor member 24, vertically to exert maximum pulling force on the sheet member 20.

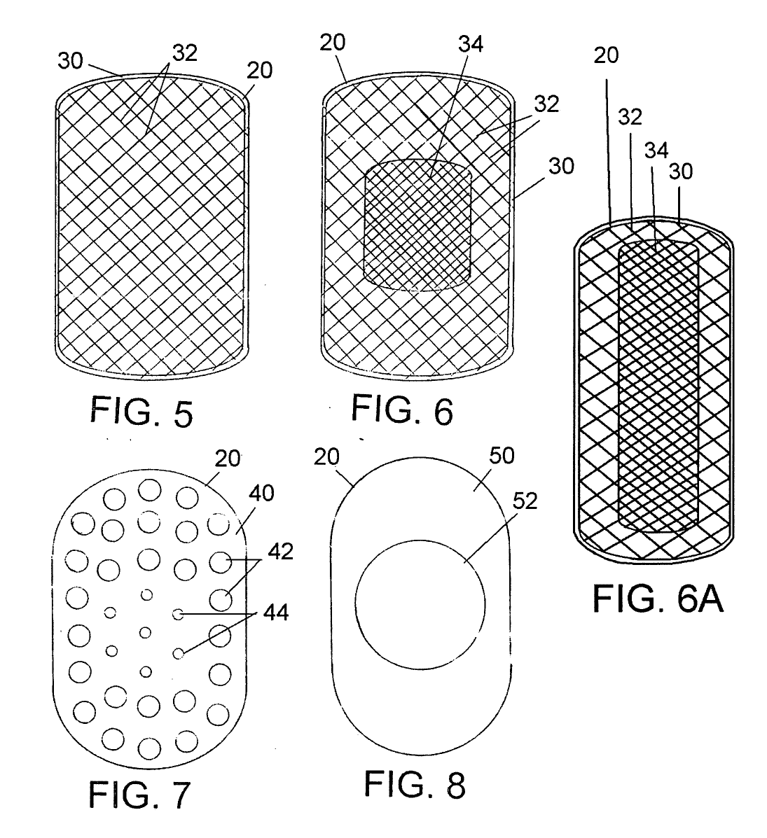

FIG. 5 illustrates an embodiment of the sheet member 20 in the form of a sheet of flexible and shapable magnetically attractable mesh material made of woven wires 32 attached to a border of framing wire 30 of the same or heavier gage. At least the wires 32, are either material that a magnet can attract, such as iron or cobalt containing metal or metal coated in plastic (nickel being usable only if it can be shielded in view of its allergy effects). Wires 32 may be magnetic material themselves to increase their attraction to attractor member 24. The border wire 30 may also be magnetically attractable or magnetic but might be non-magnetic, e.g. surgical steel, since the pulling force is best exerted near the center of the sheet member 20.

To this end, and as illustrated in FIGS. 6 and 6A, additional magnetic or magnetically attractable material is advantageously provided at the center of the sheet member were more pulling force should be concentrated. This can be done by providing an extra dense region of wire mesh 34 at the center of sheet member 20.

FIG. 7 illustrates an embodiment of the sheet member 20 in the form of a flexible and bendable perforated sheet of magnetic or magnetically attractable material 40. Again to increase the magnetic pulling force near the center, perforations 42 at the outer margins are larger and/or more densely provided than the smaller and/or more sparse perforations 44 in the central region of the sheet member 20.

FIG. 8 illustrates an embodiment of sheet member 20 in the form of a solid but thin sheet of magnetic or magnetically attractable material 50 with an central region 52 of thicker material for increasing the magnetic pulling action there.

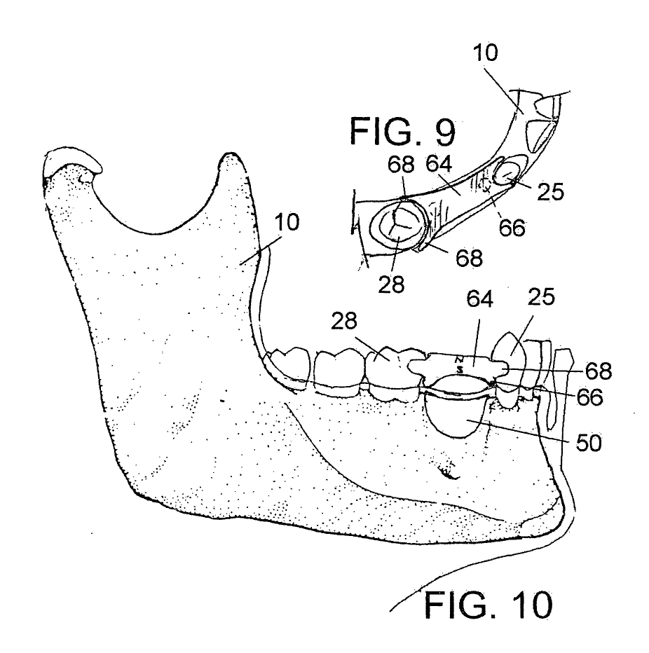

Turning to FIGS. 9 and 10, permanent magnet attractor 64 is secured to the adjacent teeth numbers 25 and 28 by a pair of flexible fingers 68 at each end. Proper spacing above the sheet member 50 to allow the outward growth of bone, is also achieved by projections 66 that extend toward the jawbone and sit on the gum to elevate the attractor 64.

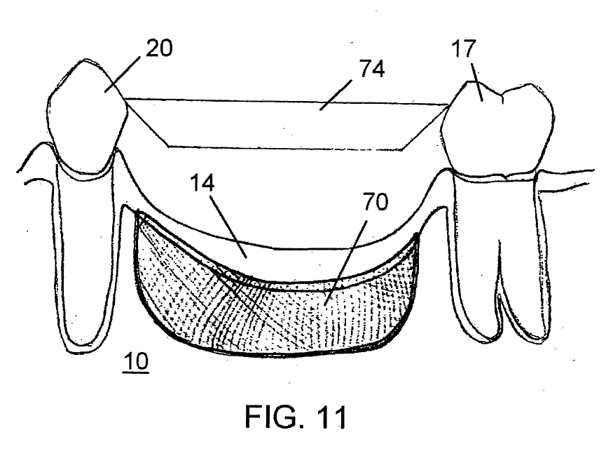

FIG. 11 is an enlarged side view of another embodiment of the apparatus of the invention showing how a permanent magnet attractor 74 is wedge-shaped and can be fixed between a pair of teeth numbers 17 and 20, and spaced over a tooth-less area of the jawbone 10 that is covered by a magnetically attractable sheet member 70 which, in turn, is covered by soft tissue or gum 14.

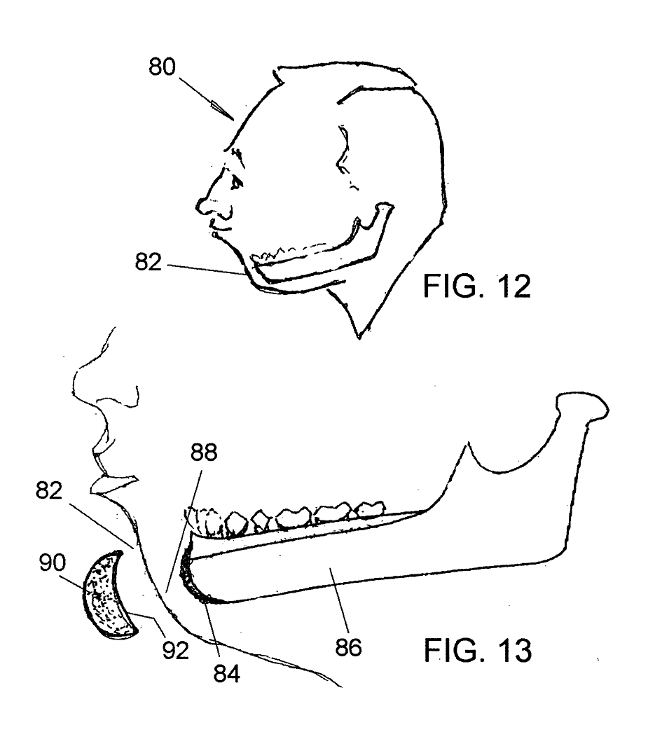

FIG. 12 shows the head 80 of a subject with a receding chin 82 who can benefit from the present invention. As shown in FIG. 13, the apparatus of the invention for improving the receding chin shown in FIG. 12, comprises a magnetically attractable sheet member 84 of mesh or other sheet structure of appropriate shape and of the types illustrated in FIGS. 5 to 8, for example, for covering the frontal surface of the mandible or jawbone 86 of the subject. Sheet member 84 is placed under the soft tissue of the chin at 88, and over the frontal surface of the mandible 86. A magnet or attractor member 90 is placed over the chin at 88, and held by any appropriate means such as a strap around the head or adhesive on the inner surface 92 of the magnet that contacted the chin at 88. After a period of time, e.g. 2 to 6 weeks, bone will grown outwardly of the front surface of the mandible 86, under the constant but gentle pulling force exerted on sheet member 84 by magnet 90, thus improving the subjects appearance.

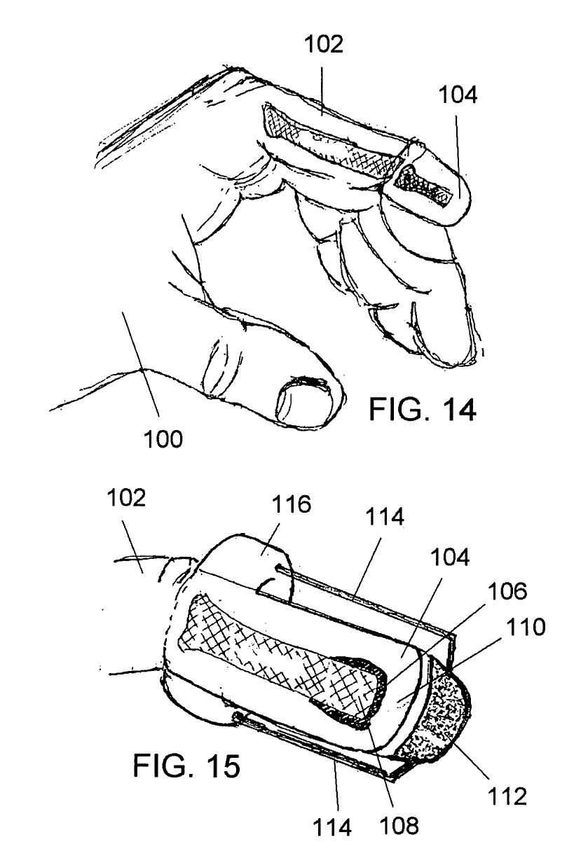

FIG. 14 shows the hand 100 of a subject who has lost the tip of his/her index finger 102 and thus has a finger stump 104. As shown in FIG. 15, the apparatus of the invention comprises a magnetically attractable sheet member 106 in the form of a cap structure of appropriate shape and of the types illustrated in FIGS. 5 to 8, for example, for covering the severed end 108 of the distal phalange of the subject's index finger 102. Sheet member 106 is placed under the soft tissue 110 that has grown over the end 108 of the finger bone, and over the surface of the bone end 108. A magnet or attractor member 112 is placed over the end of the finger stump tissue 110, and is held by any appropriate means such as a pair of rods 114 connected between the magnet 112 and a ring 116 around the finger 102 or adhesive on the inner surface of the magnet that contacted the tissue 110, for example. After a period of time, e.g. 2-6 weeks, bone will grown outwardly of the front surface of the finger bone at 108 under the constant but gentle pulling force exerted on sheet member 106 by magnet 112, thus lengthening the finger stump. This apparatus and method of the invention can be used to lengthen the damaged end of any long bone in the body that is covered by soft tissue.

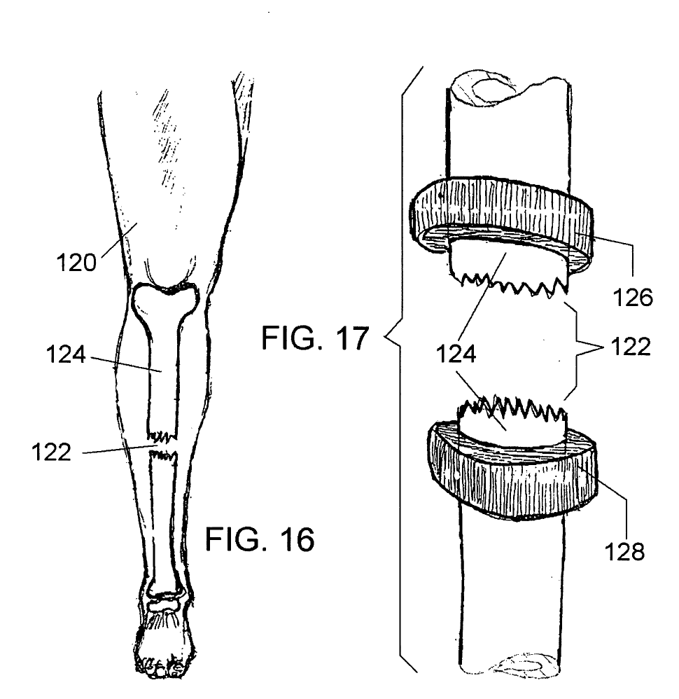

FIG. 16 is a schematic view of the leg 120 of a subject with a serious fracture 122 in the subject's tibia 124, which, if allowed to hear with conventional means may lead to shortening of the subject's leg because of the loss of bone length in the fracture area. FIG. 17 shows the apparatus of the invention for treating the fracture which comprises a pair of magnetic attractors 126 and 128 that have opposite poles facing each other across the fracture 122. Each attractor is in the form of a ring that is fixed around the broken bone adjacent the fracture, e.g. by pins, screws, biologically acceptable adhesive of other means. Both magnetic attractors are overeat bone surface and under the soft tissue of the leg. The attraction of the attractors 126, 128 to each other over a period of time, e.g. 2 to 6 weeks, will cause the bone ends to grow toward each other and once they touch, to knit together and heal the fracture without shortening the leg. Although taking advantage of the same general principle, the invention of FIG. 17 caused lengthening of the long bone on either side of a fracture by, in effected holding on to the opposite damaged ends of bone on either side of the fracture and gently but continuously pulling the ends toward each other. Growth takes place behind each magnet as the bone tissue grows to try and relieve the pulling pressure. The method and apparatus of the invention for the embodiment of FIG. 17 must also include some form of immobilization of the two parts of the long bone 124 to maintain the correct spacing in the fracture area so that the damaged bone ends have room to grow toward each other. Otherwise the muscles of the leg will tend to pull the two parts of the bone together and close the gap in the fracture area 122. A conventional plaster cast can be used for this purpose, or other immobilization means schematically depicted by the larger bracket in FIG. 17.

While specific embodiments of the invention have been shown and described in detail to illustrate the application of the principles of the invention, it will be understood that the invention may be embodied otherwise without departing from such principles.

1. A periosteal distraction osteogenesis method comprising:
applying to a surface of living bone that is covered by soft tissue, a sheet member for covering at least a portion of the bone surface under the soft tissue, at which bone growth that is outward and normal to the bone surface is desired;
securing an attractor member at an outwardly spaced location from the portion of the bone surface at which the bone growth is desired; and
magnetically attracting the sheet member and the attractor member to each other for applying a pulling force on the sheet member that is away from the portion of the bone surface at which bone growth is desired for causing growth of bone outwardly and normally to the bone surface.

2. The method of claim 1, wherein the sheet member is a sheet of magnetically attractable material and the attractor member is a permanent magnet.

3. The method of claim 1, wherein the sheet member is a sheet of magnetically attractable mesh material and the attractor member is a permanent magnet.

4. The method of claim 1, wherein the sheet member is a sheet of magnetically attractable perforated material and the attractor member is a permanent magnet.

5. The method of claim 1, wherein the sheet member is a sheet of magnetically attractable material having an increased attraction characteristic at said portion of the bone surface and less attraction characteristic around said portion of the bone surface.

6. The method of claim 1, wherein the sheet member is a sheet of magnetically attractable material and the attractor member is a permanent magnet, said portion of the bone comprising an edentulous area of a jawbone crest, the method including wrapping the edentulous area of the jawbone crest with the sheet member and securing the attractor member to at least one tooth that is adjacent the edentulous area of the jawbone crest.

7. The method of claim 1, wherein the sheet member is a sheet of saddle shaped magnetically attractable mesh material and the attractor member is a permanent magnet, said portion of the bone comprising an edentulous area of a jawbone crest, the method including wrapping the edentulous area of the jawbone crest with the sheet member and securing the attractor member to at least one tooth that is adjacent the edentulous area of a jawbone crest.

8. The method of claim 1, wherein the sheet member is a sheet of magnetically attractable material and the attractor member is a permanent magnet, said portion of the bone comprising an edentulous area of a jawbone crest, the method including wrapping the edentulous area of the jawbone crest with the sheet member and securing the attractor member across two tooth that are on opposite sides of the edentulous area of the jawbone crest, outwardly of the soft tissue which comprises gingival tissue over the edentulous area.

9. The method of claim 1, wherein the sheet member is a sheet of magnetically attractable material and the attractor member is a permanent magnet, said portion of the bone comprising a frontal surface of a mandible under tissue of a chin of a subject, the method including wrapping the frontal surface of the mandible with the sheet member and securing the attractor member over a front of the chin of the subject.

10. The method of claim 1, wherein the sheet member is a sheet of magnetically attractable material and the attractor member is a permanent magnet, said portion of the bone comprising a damaged end of a long bone of a subject having soft tissue over the damaged end, the method including wrapping the damaged end with the sheet member and securing the attractor member over the soft tissue that is over the damaged end.

11. A periosteal distraction osteogenesis apparatus comprising:
a sheet member for covering at least a portion of a surface of living bone that is covered by soft tissue, the sheet member being adapted to be under the soft tissue and bone growth outwardly and normally to the bone surface being desired;
an attractor member adapted to magnetically attract the sheet member for exerting a pulling force on the sheet member in a direction outwardly and normally of the bone surface; and
means for securing the attractor member at an outwardly spaced location from the portion of the bone surface at which the bone growth is desired for pulling on the sheet member to cause growth of bone outwardly and normally to the bone surface.

12. The apparatus of claim 11, wherein the sheet member is a sheet of magnetically attractable material and the attractor member is a permanent magnet.

13. The apparatus of claim 11, wherein the sheet member is a sheet of magnetically attractable mesh material and the attractor member is a permanent magnet.

14. The apparatus of claim 11, wherein the sheet member is a sheet of magnetically attractable perforated material and the attractor member is a permanent magnet.

15. The apparatus of claim 11, wherein the sheet member is a sheet of magnetically attractable material having an increased attraction characteristic at said portion of the bone surface and less attraction characteristic around said portion of the bone surface.

16. The apparatus of claim 11, wherein the sheet member is a sheet of magnetically attractable material and the attractor member is a permanent magnet, said portion of the bone comprising an edentulous area of a jawbone crest, the sheet member being adapted to be wrapped over the edentulous area of the jawbone crest and the means for securing comprising means for securing the attractor member to at least one tooth that is adjacent the edentulous area of the jawbone crest.

17. The apparatus of claim 11, wherein the sheet member is a sheet of saddle shaped magnetically attractable mesh material and the attractor member is a permanent magnet, said portion of the bone comprising an edentulous area of a jawbone crest, the sheet member being adapted to be wrapped over the edentulous area of the jawbone crest and the means for securing comprising means for securing the attractor member to at least one tooth that is adjacent the edentulous area of the jawbone crest.

18. The apparatus of claim 11, wherein the sheet member is a sheet of magnetically attractable material and the attractor member is a permanent magnet, said portion of the bone comprising an edentulous area of a jawbone crest, the sheet member being adapted to be wrapped over the edentulous area of the jawbone crest and the means for securing comprising means for securing the attractor member across two tooth that are on opposite sides of the edentulous area of the jawbone crest, outwardly of the soft tissue which comprises gingival tissue over the edentulous area.

19. The apparatus of claim 11, wherein the sheet member is a sheet of magnetically attractable material and the attractor member is a permanent magnet, said portion of the bone comprising a frontal surface of a mandible under tissue of a chin of a subject, the apparatus including the sheet member being adapted for wrapping the frontal surface of the mandible with the sheet member and means for securing the attractor member over a front of the chin of the subject.

20. The apparatus of claim 11, wherein the sheet member is a sheet of magnetically attractable material and the attractor member is a permanent magnet, said portion of the bone comprising a damaged end of a long bone of a subject having soft tissue overeat damaged end, the apparatus comprising the sheet member being adapted for wrapping the damaged end with the sheet member and means for securing the attractor member over the soft tissue that is over the damaged end.

21. An osteogenesis method comprising:
applying to a surface of a damaged end of a living long bone on one side of a fracture covered by soft tissue, a first attractor member;
applying to a surface of a damaged end of a living long bone on an opposite side of the fracture covered by soft tissue, a second attractor member;
immobilizing the bone on both sides of the fracture; and
magnetically attracting the first and second attractor members toward each other for applying a pulling force on each damaged end for causing bone growth toward the fracture from both sides of the fracture.

22. An osteogenesis apparatus comprising:
a first attractor member for being applied to a surface of a damaged end of a living long bone on one side of a fracture covered by soft tissue;
a second attractor member for being applying to a surface of a damaged end of a living long bone on an opposite side of the fracture covered by soft tissue;
means for immobilizing the bone on both sides of the fracture; and
means for magnetically attracting the first and second attractor members toward each other for applying a pulling force on each damaged end for causing bone growth toward the fracture from both sides of the fracture.
